(19) United States
(12) Patent Application Publication
(10) Pub. No.: US 2014/0057006 A1
(43) Pub. Date: Feb. 27, 2014

(54) Pharmaceutical Compositions for the Treatment of Warts

(71) Applicant: ATP Marketing & Promotion AG, Niederurnen (CH)

(72) Inventor: Mario Dominik Nicolaas Oldani, Ameide (NL)

(73) Assignee: ATP Marketing & Promotion AG, Niederurnen (CH)

(21) Appl. No.: 14/029,031

(22) Filed: Sep. 17, 2013

Related U.S. Application Data
(63) Continuation of application No. 12/992,166, filed on Jan. 24, 2011.

(30) Foreign Application Priority Data
May 15, 2008 (NL) NL1035426

(51) Int. Cl. A61K 31/695; A61K 36/14; A61K 36/81; A61K 36/53; A61K 36/185; A61K 36/54; A61K 36/736; A61K 36/61

(52) U.S. Cl. CPC A61K 31/695; A61K 36/736; A61K 36/14; A61K 36/61; A61K 36/53; A61K 36/185; A61K 36/54; A61K 36/81
USPC 424/735; 424/770; 424/769; 424/745; 424/725; 424/739; 424/760

(57) ABSTRACT

The present invention relates to pharmaceutical compositions for the treatment, and especially topical treatment, of warts and the use thereof for the treatment of warts. Specifically, the present invention relates to pharmaceutical compositions for the treatment of warts, comprising: a) 0.1 to 10 weight % dimethicone; b) 5 to 25 weight % of one or more vegetable oils; c) one or more pharmaceutically acceptable excipients and/or carriers; d) 2 to 6 weight % urea; and e) water up to 100 weight %.

### CROSS-REFERENCE TO RELATED APPLICATIONS

This application is a continuation of co-pending U.S. patent application Ser. No. 12/992,166, filed Jan. 24, 2011, which is a U.S. National Phase Application filed under 35 U.S.C. §371 claiming priority to PCT Application No. PCT/EP2009/055904, filed May 15, 2009, and which claims priority to Netherlands Patent Application No. NL1035426, filed May 15, 2008, each of which is incorporated by reference herein in their entirety.

### BACKGROUND OF THE INVENTION

The present invention relates to pharmaceutical compositions for the treatment, and especially the topical treatment, of warts and the use thereof for the treatment of warts.

A wart (Verrucca), is a hard, or horny, callous deformity of the upper skin generally in the form of a cauliflower-like nodule. At least four different types of warts are known: the common wart (Verruca vulgaris), the plantar wart (Verucca plantaris), the flat wart (Verucca plana) and the filiform wart (Verucca filiformis).

Warts generally comprise a core of connective tissue, and comprised therein blood vessels, coated with one or more layers of epithelial tissue. Warts can occur alone or in groups.

Warts are generally caused by human papilloma viruses. Papilloma virus infected skin cells grow faster than non-infected skin cells. This growth difference, in comparison with the surrounding skin cells, results in a wart. Because of their virulent nature, warts are contagious.

Although in most cases warts do not cause other adverse medical conditions, and even spontaneously disappear after a certain time period, individuals receiving organ transplants, or other individuals with a compromised immune system, often develop warts on their hands and lower arms which warts sometimes appear to develop into squamous cell carcinomas.

For the treatment of warts, several methods are known from the prior art.
1) Application of podophyllum resin paint [podophyllum resin I.P.'66 (20% w/v), benzoin I.P. (10% w/v), aloes I.P. (2% w/v), isopropyl alcohol I.P. to make (100% v/v)];
2) Keratolysis, the removal of dead surface skin cells usually using salicylic acid, blistering agents, immune system modifiers (“immunomodulators”), or formaldehyde, often with mechanical paring of the wart with a pumice stone, blade etc.;
3) Cryosurgery, which involves freezing the wart (generally with liquid nitrogen), creating a blister between the wart and epidermal layer, after which the wart and surrounding dead skin falls off. An average of 3 to 4 treatment is required for warts of thin skin. Warts on calloused skin like plantar warts might take dozens or more treatments.
4) Surgical curettage of the wart;
5) Laser treatment, often with a pulse dye laser or carbon dioxide (CO₂) laser. Pulse dye lasers work by selective absorption by blood cells (specifically haemoglobin). CO₂ lasers work by selective absorption by water molecules. Pulse dye lasers are less destructive and more likely to heal without scarring. CO₂ laser works by vaporizing and destroying tissue and skin Both laser treatments can be painful, expensive, and can cause scarring. CO₂ lasers will require local anaesthetic, while pulse dye lasers might need conscious sedation. The therapy comprises 1 to 4 treatments.
6) Infrared coagulator, an intense source of infrared light in a small beam like a laser. This works essentially on the same principle as laser treatment. Like the laser, it can cause blistering, pain and scarring.
7) Imiquimod, a topical cream that helps the body's immune system combat the wart virus by encouraging interferon production.
8) Injection of Candida, mumps, or Trichophyton antigens at the site of the wart, which stimulates the body's immune system.
9) Cantharidin, a chemical found naturally in many members of the beetle family Meloidae which causes dermal blistering. Either used by itself or compounded with podophyllin.
10) Bleomycin, one or two injections are used. Bleomycin can cause necrosis of digits and Raynaud syndrome.
11) Dinitrochlorobenzene (DNCB), like salicylic acid, this is applied directly onto the wart. Studies showed this method was effective with a cure rate of 80% compared to 38% for a placebo. But DNCB must be used much more cautiously than salicylic acid; the chemical is a known mutagen and, therefore, capable of causing genetic mutations. This drug induces an allergic immune response resulting in inflammation that wards off the wart-causing virus.
12) Fluorouracil, which inhibits DNA synthesis, is being used as an experimental treatment. It is applied directly to the wart (especially plantar warts) and covered with, for example, tape. This treatment is combined with the use of a pumice stone, but tends to work very slowly.
   However, none of the above mentioned methods provide an effective, or satisfying treatment of warts.

### SUMMARY OF THE INVENTION

It is an object of the present invention to provide novel pharmaceutical compositions for the treatment, and especially the topical treatment, of warts.

This object, amongst other objects, is met by the pharmaceutical compositions for the treatment of warts as defined herein.

Specifically, this object, amongst other objects, is met by pharmaceutical compositions for the treatment of warts comprising:
a) 0.1 to 10 weight % dimethicone;
b) 5 to 25 weight % of one or more vegetable oils;
c) one or more pharmaceutically acceptable excipients and/or carriers;
d) 2 to 6 weight % urea; and
e) water up to 100 weight %.

### DETAILED DESCRIPTION OF THE PREFERRED EMBODIMENTS

As used herein, the term weight % relates to the weight of the indicated ingredient divided by the weight of the total pharmaceutical composition and multiplied by 100%.

Dimethicone (polydimethylsolixane, PDMS) is a flexible silicon polymer generally used in hand lotions and cosmetic products. The present inventor surprisingly discovered that besides this known use of dimethicone, dimethicone also has a beneficial effect on the treatment of warts.

The pharmaceutical compositions according to the present invention comprise urea. Urea, because of its water retention (wetting) characteristics is one of the most important water retention compounds of the skin.

Besides dimethicone and urea, the present pharmaceutical compositions also beneficially comprise one or more vegetable oils and one or more pharmaceutically acceptable excipients and/or carriers.

In a preferred embodiment of the present invention, the one or more vegetable oils are chosen from the group consisting of almond oil, tea tree oil, lavender oil, geranium oil, lemon oil, thuja oil, and cinnamon oil.

Almond oil, for example, beneficially regulates the moisture content of the skin and reduces skin irritations.

Lavender oil, for example, beneficially provides disinfecting, bactericide, pain killing, curing and purifying characteristics.

Geranium oil, for example, beneficially provides infection inhibiting, fungus inhibiting and wound healing characteristics.

Tea tree oil, for example, beneficially provides bacterial, viral and fungal inhibiting growth characteristics. Further, tea tree oil is pain and scar reducing, and improves healing. Furthermore, tea tree oil readily mixes with natural skin oils allowing a quick effect on the source of infection.

Cinnamon oil, for example, aids in combating bacteria, viruses and fungi, is inflammation inhibiting and improves the blood flow.

Thuja oil, for example, is a bactericide and fungicide.

Lemon oil, for example, is disinfecting.

According to a preferred embodiment, the present pharmaceutical composition further comprises 0.5 to 2 weight % red pepper powder. Red pepper, also designated as Chilean or Spanish pepper or hot pepper, is rich in vitamin C, reduces pain, stimulates nerves, and is disinfecting.

Preferably, the present one or more excipients and/or carriers are chosen from the group consisting of ammonium acryloyldimethyl taurate/VP copolymer, sodium benzoate, potassium sorbate and citric acid.

According to an especially preferred embodiment of the present invention, the pharmaceutical compositions comprise:
a) 0.5 to 3 weight % dimethicone;
b) vegetable oils comprising:
   1) 1 to 3 weight % almond oil;
   2) 1 to 3 weight % tea tree oil;
   3) 1 to 2 weight % lavender oil;
   4) 1 to 2 weight % geranium oil;
   5) 0.5 to 2 weight % lemon oil;
   6) 0.5 to 2 weight % thuja oil;
   7) 0.5 to 2 weight % cinnamon oil.
c) pharmaceutically acceptable excipients and/or carriers comprising:
   1) 0.5 to 3 weight % ammonium acryloyldimethyl taurate/VP copolymer;
   2) 0.1 to 0.5 weight % sodium benzoate;
   3) 0.05 to 0.25 weight % potassium sorbate;
   4) 0.05 to 0.25 weight % citric acid;
d) 2 to 6 weight % urea;
e) 0.5 to 3 weight % red pepper powder;
f) water up to 100 weight %.

According to a most preferred embodiment of the present invention, the pharmaceutical compositions comprise:
a) 1 weight % dimethicone;
b) vegetable oils comprising:
   1) 2 weight % almond oil;
   2) 2 weight % tea tree oil;
   3) 1.5 weight % lavender oil;
   4) 1.5 weight % geranium oil;
   5) 1 weight % lemon oil;
   6) 1 weight % thuja oil;
   7) 1 weight % cinnamon oil.
c) pharmaceutically acceptable excipients and/or carriers comprising:
   1) 1.65 weight % ammonium acryloyldimethyl taurate/VP copolymer;
   2) 0.3 weight % sodium benzoate;
   3) 0.16 weight % potassium sorbate;
   4) 0.15 weight % citric acid;
d) 4 weight % urea;
e) 1 weight % red pepper powder;
f) water up to 100 weight %.

The pharmaceutical compositions according to the present invention are, in a preferred embodiment, formulated for topical administration, preferably in the form of an ointment, crème or tincture.

The present pharmaceutical compositions are especially effective for the treatment of warts. Accordingly, the present invention also relates to dimethicone for the topical treatment of warts and the use of dimethicone for the preparation of a medicament for the topical treatment of warts.

A beneficial effect of the present pharmaceutical compositions, and the use thereof, is further provided by the observation that the wart surrounding skin substantially remains unaffected by the present compositions.

The present pharmaceutical compositions are suitably, for example and preferably, topically applied, 1 to 5 times a day, preferably 1 to 2 times a day, on the wart. After application, the present pharmaceutical composition is massaged in and allowed to be absorbed. The indicated treatment is repeated until the wart has at least visibly disappeared.

The indicated treatment regime using the present compositions has been used by a number of individuals repeatedly suffering from warts and a significant subset of these individuals reported the disappearance of the warts within a time period of 1 week to several months without pain, scarring or any other side effects.

1. A method of treating warts, comprising:
applying a topical medicament to at least one wart, the medicament comprising:
a) 0.1 to 10 weight % dimethicone;
b) 5 to 25 weight % of one or more vegetable oils;
c) one or more pharmaceutically acceptable excipients and/or carriers;
d) 2 to 6 weight % urea; and
e) water up to 100 weight %.

2. The method according to claim 1, wherein said one or more vegetable oils of said medicament are chosen from the group consisting of almond oil, tea tree oil, lavender oil, geranium oil, lemon oil, thuja oil, and cinnamon oil.

3. The method according to claim 1, the medicament further comprising:
f) 0.5 to 2 weight % red pepper powder.

4. The method according to claim 1, wherein said one or more pharmaceutically acceptable excipients and/or carriers of said medicament are chosen from the group consisting of ammonium acryloyldimethyl taurate/VP copolymer, sodium benzoate, potassium sorbate and citric acid.

5. The method according to claim 1, wherein the medicament comprises:
a) 0.5 to 3 weight % dimethicone;
b) vegetable oils comprising:
1) 1 to 3 weight % almond oil;
2) 1 to 3 weight % tea tree oil;
3) 1 to 2 weight % lavender oil;
4) 1 to 2 weight % geranium oil;
5) 0.5 to 2 weight % lemon oil;
6) 0.5 to 2 weight % thuja oil; and
7) 0.5 to 2 weight % cinnamon oil;
c) pharmaceutically acceptable excipients and/or carriers comprising:
1) 0.5 to 3 weight % ammonium acryloyldimethyl taurate/ VP copolymer;
2) 0.1 to 0.5 weight % sodium benzoate;
3) 0.05 to 0.25 weight % potassium sorbate;
4) 0.05 to 0.25 weight % citric acid;
d) 2 to 6 weight % urea;
e) 0.5 to 3 weight % red pepper powder; and
f) water up to 100 weight %.

6. The method according to claim 1, wherein the medicament comprises:
a) 1 weight % dimethicone;
b) vegetable oils comprising:
1) 2 weight % almond oil;
2) 2 weight % tea tree oil;
3) 1.5 weight % lavender oil;
4) 1.5 weight % geranium oil;
5) 1 weight % lemon oil;
6) 1 weight % thuja oil; and
7) 1 weight % cinnamon oil;
c) pharmaceutically acceptable excipients and/or carriers comprising:
1) 1.65 weight % ammonium acryloyldimethyl taurate/VP copolymer;
2) 0.3 weight % sodium benzoate;
3) 0.16 weight % potassium sorbate; and
4) 0.15 weight % citric acid;
d) 4 weight % urea;
e) 1 weight % red pepper powder; and
f) water up to 100 weight %.

7. The method according to claim 1, wherein the medicament is applied to the at least one wart 1 to 5 times per day.

8. The method according to claim 7, wherein the medicament is applied to the at least one wart for at least one week.

9. A method of treating warts, comprising:
applying a topical medicament to at least one wart, the medicament comprising:
a) 0.5 to 3 weight % dimethicone;
b) vegetable oils comprising:
1) 1 to 3 weight % almond oil;
2) 1 to 3 weight % tea tree oil;
3) 1 to 2 weight % lavender oil;
4) 1 to 2 weight % geranium oil;
5) 0.5 to 2 weight % lemon oil;
6) 0.5 to 2 weight % thuja oil; and
7) 0.5 to 2 weight % cinnamon oil;
c) pharmaceutically acceptable excipients and/or carriers comprising:
1) 0.5 to 3 weight % ammonium acryloyldimethyl taurate/ VP copolymer;
2) 0.1 to 0.5 weight % sodium benzoate;
3) 0.05 to 0.25 weight % potassium sorbate; and
4) 0.05 to 0.25 weight % citric acid;
d) 2 to 6 weight % urea;
e) 0.5 to 3 weight % red pepper powder; and
f) water up to 100 weight %;
wherein said medicament is one of an ointment, crème, or tincture.

10. A topical medicament comprising:
a) 0.5 to 3 weight % dimethicone;
b) vegetable oils comprising:
1) 1 to 3 weight % almond oil;
2) 1 to 3 weight % tea tree oil;
3) 1 to 2 weight % lavender oil;
4) 1 to 2 weight % geranium oil;
5) 0.5 to 2 weight % lemon oil;
6) 0.5 to 2 weight % thuja oil; and
7) 0.5 to 2 weight % cinnamon oil;
c) pharmaceutically acceptable excipients and/or carriers comprising:
1) 0.5 to 3 weight % ammonium acryloyldimethyl taurate/VP copolymer;
2) 0.1 to 0.5 weight % sodium benzoate;
3) 0.05 to 0.25 weight % potassium sorbate;
4) 0.05 to 0.25 weight % citric acid;
d) 2 to 6 weight % urea;
e) 0.5 to 3 weight % red pepper powder; and
f) water up to 100 weight %.

11. The medicament according to claim 10, wherein the medicament comprises:
a) 1 weight % dimethicone;
b) vegetable oils comprising:
1) 2 weight % almond oil;
2) 2 weight % tea tree oil;
3) 1.5 weight % lavender oil;
4) 1.5 weight % geranium oil;
5) 1 weight % lemon oil;
6) 1 weight % thuja oil; and
7) 1 weight % cinnamon oil;
c) pharmaceutically acceptable excipients and/or carriers comprising:
1) 1.65 weight % ammonium acryloyldimethyl taurate/VP copolymer;
2) 0.3 weight % sodium benzoate;
3) 0.16 weight % potassium sorbate;
4) 0.15 weight % citric acid;
d) 4 weight % urea;
e) 1 weight % red pepper powder; and
f) water up to 100 weight %.
